(19) United States
(12) Patent Application Publication
(10) Pub. No.: US 2026/0265174 A1
(43) Pub. Date: Sep. 10, 2026

(54) PROCESS FOR PREPARING POTASSIUM IRON(III) ETHYLENEDIAMINETETRAACETIC ACID COMPLEX SALT

(71) Applicant: BASF SE, Ludwigshafen (DE)

(72) Inventors: Frank BACHMANN, Ludwigshafen (DE); Doris KREMZOW-GRAW, Ludwigshafen (DE); Paul KLINGELHOEFER, Mannheim (DE); Imme WITTE, Ludwigshafen (DE); Peter OECHSLE, Ludwigshafen (DE); Sarah Veronika ILLIES, Ludwigshafen (DE)

(21) Appl. No.: 19/472,125

(22) PCT Filed: Apr. 17, 2024
(86) PCT No.: PCT/EP24/60387

§ 371 Date: Oct. 3, 2025

(30) Foreign Application Priority Data
Apr. 18, 2023 (EP) 23168404.4

(51) Int. Cl. C07C 227/18

(52) U.S. Cl. CPC C07C 227/18

(57) ABSTRACT

Disclosed herein is a process for preparing the potassium salt of iron(III) ethylenediaminetetraacetic acid complex (KFe(III)EDTA) by partially neutralizing the acid form of EDTA with a basic potassium salt, reacting the partially neutralized EDTA with a Fe₃O₄ source to a potassium iron ethylenediaminetetraacetic acid complex salt containing both iron (II) and (III), oxidizing the iron(II) contained therein with an oxygen-based oxidation agent, and adjusting the pH to from 5 to 7 with a basic potassium salt.

The present invention relates to a process for preparing the potassium salt of iron(III) ethylenediaminetetraacetic acid complex (abbreviated as KFe(III)EDTA) by partially neutralizing the acid form of EDTA with a basic potassium salt, reacting the partially neutralized EDTA with a Fe₃O₄ source to a potassium iron ethylenediaminetetraacetic acid complex salt containing both iron (II) and (III), oxidizing the iron(II) contained therein with an oxygen-based oxidation agent, and adjusting the pH to from 5 to 7 with a basic potassium salt.

### TECHNICAL BACKGROUND

Iron is involved in the synthesis of chlorophyll and in other enzymatic and metabolic processes in plants. Iron deficiency in plants is generally not caused by a lack of iron in the soil, but is rather due to its low availability in a form that can be absorbed by the plant. Iron availability increases with decreasing soil pH, but changing the soil pH is neither an easy undertaking in practice nor desirable in most cases. In agricultural practice, iron is generally made available to the plants by applying iron chelates as iron source.

Among the various iron complexes, iron chelates of the potassium salt of EDTA are used as speciality fertilizer. While the analogous sodium salt is also useful for providing the plants with iron in well-available form, the potassium salt has the advantage of having a higher dissolution rate and moreover of additionally supplying potassium, which, in contrast to sodium, is a primary plant (macro)nutrient (used for example in NPK fertilizers), while sodium just contributes to undesired salination and is known to be phytotoxic. The potassium salt of EDTA-chelated iron can thus also be used in hydroponic cultures, in greenhouses and in areas where (further) salination is to be avoided.

Various synthetic methods for the preparation of salts of EDTA iron chelates are described in the art.

DE 2107079 relates to a process for preparing aqueous solutions of ammonium salts of Fe(III) aminopolycarboxylic complexes. To this purpose, an iron oxide containing iron(III) is reacted with an aminopolycarboxylic acid, such as EDTA, partially neutralized with ammonia or a water-soluble amine, in aqueous medium under heating to 82-104° C., and then neutralized with ammonia or an amine. An oxidation step is not mentioned. Transferring the reaction conditions applied therein to the preparation of the corresponding potassium salt does however not yield KFe(III)EDTA in satisfactory yields and with the desired specifications and properties.

EP 0471583 A1 relates to a process for the preparation of salts of iron amino and hydroxy carboxylic acid complexes by reacting an amino or hydroxy carboxylic acid or a salt or partial salt thereof with an iron oxide and a base in the presence of a ferrous (Fe(II)) salt or metallic iron or the salt of the ferrous complex to be produced as a catalyst, neutralizing the reaction product and if desired oxidizing any ferrous iron to ferric iron. EDTA is named as a suitable aminocarboxylic acid. Preferred catalyst is ferrous sulfate hydrate. Preferably, ferric ammonium EDTA is prepared, using ammonium as base, but ferric sodium EDTA and ferric potassium EDTA are said to be obtainable analogously by using NaOH or KOH, respectively. As suitable iron oxide, magnetite and α- and γ hydrated iron oxides [Fe(OH)O] are mentioned. The process for preparing ferric ammonium EDTA is preferably carried out by first mixing water, iron oxide and base, followed by the addition of the carboxylic acid. The order of addition is emphasized to be important. After reaching the desired reaction temperature, the catalyst is added. The reaction product is neutralized with ammonia and air-oxidized at about 30° C. to give Fe(III)NH₄EDTA, which is further converted to (EDTA(NH₄)₂FeOH) by the addition of more ammonia. The use of catalysts is however disadvantageous for obtaining products with no foreign ions, since these have to be removed, thus requiring further, partially tedious purification steps.

There is a constant need for efficient synthetic methods for the preparation of iron chelates of the potassium salt of EDTA, especially of such with high iron(III) content, since iron in this oxydation stage is better available to the plants. It was thus the object of the present invention to provide an efficient process for the preparation of an iron chelate of the potassium salt of EDTA with high iron(III) content. The total iron content in the obtained potassium iron chelate product should be at least 13% by weight, relative to the total weight of the product (in case of pure KFe(III)EDTA the Fe content is 14.57% by weight, relative to the total weight of KFe(III)EDTA), and the content of Fe²⁺ should be below 1% by weight, relative to the total weight of the product. Moreover, no foreign ions, and especially no sulfate, chloride or nitrate anions, should be involved, so that the obtained product is devoid of these anions without any additional step in which such anions would otherwise have to be removed.

The problem is solved by the process of the invention described in the following.

### SUMMARY OF THE INVENTION

The present invention relates to a process for preparing potassium iron(III) ethylenediaminetetraacetic acid complex salt (abbreviated as KFe(III)EDTA), comprising
(i) reacting in an aqueous medium the acid form of ethylenediaminetetraacetic acid with a basic potassium salt to a partially neutralized ethylenediaminetetraacetic acid;
(ii) reacting the reaction mixture obtained in step (i) with a Fe₃O₄ source to a potassium iron ethylenediaminetetraacetic acid complex salt containing both iron(II) and iron(III);
(iii) oxidizing the potassium iron ethylenediaminetetraacetic acid complex salt obtained in step (ii) [and containing both iron(II) and iron(III)] to potassium iron(III) ethylenediaminetetraacetic acid complex salt with an oxygen-based oxidation agent; and
(iv) adjusting the reaction mixture obtained step (iii) to a pH of from 5 to 7 with a basic potassium salt.

### DETAILED DESCRIPTION OF THE INVENTION

### Definitions

The potassium iron(III) ethylenediaminetetraacetic acid complex salt (abbreviated as KFe(III)EDTA), also termed as potassium salt of iron(III) ethylenediaminetetraacetic acid complex, is a complex with iron(III) as central metal and EDTA as chelate ligand.

The carboxylic groups of EDTA are neutralized, i.e. they are present as carboxylate groups, the counter cations being Fe(III) and K⁺.

KFe(III)EDTA can be schematically depicted as follows:

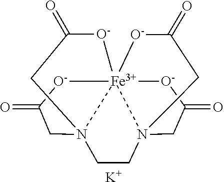

This is a simplified, idealized view of the KFe(III)EDTA product as obtained with the method of the invention, since this could, for example, be present as a hydrate, the central metal being thus complex-bound by a water molecule. Moreover, if neutralization in step (iv) is not carried out completely, a (small) part of the carboxylate groups may be present in acid form. Furthermore, if oxidation in step (iii) is not complete, a very small part (generally <1%) of the central metal may be Fe(II) (the complex salt in this case can be depicted in idealized form similarly to that of KFe(III)EDTA shown above, the difference being Fe²⁺ as central metal and the presence two K⁺ counter cations).

### Acid Form of Ethylenediaminetetraacetic Acid Means EDTA in Non-Neutralized Form, i.e. as Tetraacid:

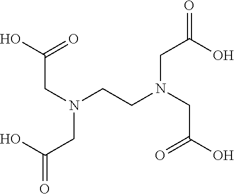 [SMILES: O=C(O)CN(CCN(CC(=O)O)CC(=O)O)CC(=O)O]

### Embodiments (E.x) of the Invention

General and preferred embodiments E.x are summarized in the following, non-exhaustive list. Further preferred embodiments become apparent from the paragraphs following this list.

E.1. A process for preparing potassium iron(III) ethylenediaminetetraacetic acid salt KFe(III)EDTA, comprising
(i) reacting in an aqueous medium the acid form of ethylenediaminetetraacetic acid with a basic potassium salt to a partially neutralized ethylenediaminetetraacetic acid;
(ii) reacting the reaction mixture obtained in step (i) with a Fe₃O₄ source to a potassium iron ethylenediaminetetraacetic acid salt containing both iron (II) and (III);
(iii) oxidizing the potassium iron ethylenediaminetetraacetic acid salt containing both iron (II) and (III) to potassium iron(III) ethylenediaminetetraacetic acid salt with an oxygen-based oxidation agent; and
(iv) adjusting the reaction mixture obtained step (iii) to a pH of from 5 to 7 with a basic potassium salt.

E.2. The process according to embodiment E.1, where in step (i) the basic potassium salt is used in an amount of from 0.7 to 1 mol per mol of the acid form of ethylenediaminetetraacetic acid, where the amount of the basic potassium salt refers to the amount of potassium in said salt.

E.3. The process according to embodiment E.2, where in step (i) the basic potassium salt is used in an amount of from 0.7 to 0.9 mol per mol of the acid form of ethylenediaminetetraacetic acid, where the amount of the basic potassium salt refers to the amount of potassium in said salt.

E.4. The process according to embodiment E.3, where in step (i) the basic potassium salt is used in an amount of from 0.75 to 0.85 mol per mol of the acid form of ethylenediaminetetraacetic acid, where the amount of the basic potassium salt refers to the amount of potassium in said salt.

E.5. The process according to embodiment E.4, where in step (i) the basic potassium salt is used in an amount of from 0.80 to 0.82 mol per mol of the acid form of ethylenediaminetetraacetic acid, where the amount of the basic potassium salt refers to the amount of potassium in said salt.

E.6. The process according to any of the preceding embodiments, where the partially neutralized ethylenediaminetetraacetic acid obtained in step (i) is the monopotassium salt of ethylenediaminetetraacetic acid or a mixture of the monopotassium salt of ethylenediaminetetraacetic acid and the acid form of ethylenediaminetetraacetic acid.

E.7. The process according to any of the preceding embodiments, where in step (i), a basic potassium salt is added to a suspension of the acid form of ethylenediaminetetraacetic acid in water.

E.8. The process according to embodiment E.7, where the suspension of the acid form of ethylenediaminetetraacetic acid in water contains 0.1 to 10 mol of the acid form of ethylenediaminetetraacetic acid per kg of water.

E.9. The process according to embodiment E.8, where the suspension of the acid form of ethylenediaminetetraacetic acid in water contains 0.5 to 5 mol of the acid form of ethylenediaminetetraacetic acid per kg of water.

E.10. The process according to embodiment E.9, where the suspension of the acid form of ethylenediaminetetraacetic acid in water contains 1 to 5 mol of the acid form of ethylenediaminetetraacetic acid per kg of water.

E.11. The process according to embodiment E.10, where the suspension of the acid form of ethylenediaminetetraacetic acid in water contains 2 to 4 mol of the acid form of ethylenediaminetetraacetic acid per kg of water.

E.12. The process according to embodiment E.7, where the suspension of the acid form of ethylenediaminetetraacetic acid in water contains 2 to 10 mol of the acid form of ethylenediaminetetraacetic acid per kg of water.

E.13. The process according to embodiment E.12, where the suspension of the acid form of ethylenediaminetetraacetic acid in water contains 2 to 5 mol of the acid form of ethylenediaminetetraacetic acid per kg of water.

E.14. The process according to embodiment E.13, where the suspension of the acid form of ethylenediaminetetraacetic acid in water contains 3 to 5 mol of the acid form of ethylenediaminetetraacetic acid per kg of water.

E.15. The process according to embodiment E.14, where the suspension of the acid form of ethylenediaminetetraacetic acid in water contains 3 to 4 mol of the acid form of ethylenediaminetetraacetic acid per kg of water.

E.16. The process according to any of embodiments E.7 to E.15, where the basic potassium salt is added as an aqueous solution.

E.17. The process according to any of the preceding embodiments, where the basic potassium salt used in steps (i) and (iv) is selected from potassium hydroxide (KOH), potassium carbonate (K₂CO₃), potassium oxide and mixtures thereof.

E.18. The process according to embodiment E.17, where the basic potassium salt used in steps (i) and (iv) is KOH.

E.19. The process according to any of the preceding embodiments, where the Fe₃O₄ source is natural or synthetic magnetite.

E.20. The process according to embodiment E.19, where the Fe₃O₄ source is natural magnetite.

E.21. The process according to any of the preceding embodiments, where the Fe₃O₄ source is used in an amount of from 0.2 to 0.5 mol per mol of the acid form of ethylenediaminetetraacetic acid (i.e. per mol of the amount of EDTA as used in step (i)), where the amount of the Fe₃O₄ source refers to the amount of Fe₃O₄ contained in said source.

E.22. The process according to embodiment E.21, where the Fe₃O₄ source is used in an amount of from 0.2 to 0.4 mol per mol of the acid form of ethylenediaminetetraacetic acid, where the amount of the Fe₃O₄ source refers to the amount of Fe₃O₄ contained in said source.

E.23. The process according to embodiment E.22, where the Fe₃O₄ source is used in an amount of from 0.30 to 0.35 mol per mol of the acid form of ethylenediaminetetraacetic acid, where the amount of the Fe₃O₄ source refers to the amount of Fe₃O₄ contained in said source.

E.24. The process according to embodiment E.23, where the Fe₃O₄ source is used in an amount of ca. 0.33 mol per mol of the acid form of ethylenediaminetetraacetic acid, where the amount of the Fe₃O₄ source refers to the amount of Fe₃O₄ contained in said source.

E.25. The process according to any of the preceding embodiments, where step (ii) is carried out at a temperature of from 80 to 100° C.

E.26. The process according to embodiment E.25, where step (ii) is carried out at a temperature of from 90 to 100° C.

E.27. The process according to any of the preceding embodiments, where step (ii) is carried out at a pH of from 3 to 5.

E.28. The process according to embodiment E.27, where step (ii) is carried out at a pH of from 3 to 4.5.

E.29. The process according to any of the preceding embodiments, where step (iii) is carried out at a pressure of from atmospheric pressure to 15 bar (1.5 MPa).

E.30. The process according to embodiment E.29, where step (iii) is carried out at a pressure of from atmospheric pressure to 10 bar (1 MPa).

E.31. The process according to embodiment E.30, where step (iii) is carried out at a pressure of from atmospheric pressure to 6 bar (0.6 MPa).

E.32. The process according to embodiment E.31, where step (iii) is carried out at a pressure of from atmospheric pressure to 5 bar (0.5 MPa).

E.33. The process according to embodiment E.32, where step (iii) is carried out at atmospheric pressure.

E.34. The process according to embodiment E.30, where step (iii) is carried out at a pressure of from 3 to 10 bar (0.3 to 1 MPa).

E.35. The process according to embodiment E.34, where step (iii) is carried out at a pressure of from 3 to 6 bar (0.3 to 0.6 MPa).

E.36. The process according to embodiment E.35, where step (iii) is carried out at a pressure of from 3 to 5 bar (0.3 to 0.5 MPa).

E.37. The process according to any of the preceding embodiments, where the oxygen-based oxidation agent used in step (iii) is selected from oxygen, air, oxygen/nitrogen mixtures different from air, and hydrogen peroxide.

E.38. The process according to embodiment E.37, where the oxygen-based oxidation agent used in step (iii) is selected from oxygen, air and hydrogen peroxide.

E.39. The process according to embodiment E.38, where the oxygen-based oxidation agent used in step (iii) is selected from oxygen and air.

E.40. The process according to embodiment E.39, where the oxygen-based oxidation agent used in step (iii) is air.

E.41. The process according to any of the preceding embodiments, where step (iii) is carried out at a temperature of from 10 to 100° C.

E.42. The process according to embodiment E.41, where step (iii) is carried out at a temperature of from 10 to 40° C. (especially if the oxygen-based oxidation agent used in step (iii) is selected from oxygen, air and oxygen/nitrogen mixtures different from air).

E.43. The process according to embodiment E.42, where step (iii) is carried out at a temperature of from 15 to 30° C. (especially if the oxygen-based oxidation agent used in step (iii) is selected from oxygen, air and oxygen/nitrogen mixtures different from air).

E.44. The process according to embodiment E.43, where step (iii) is carried out at a temperature of from 15 to 25° C. (especially if the oxygen-based oxidation agent used in step (iii) is selected from oxygen, air and oxygen/nitrogen mixtures different from air).

E.45. The process according to embodiment E.44, where step (iii) is carried out at a temperature of from 20 to 25° C. (especially if the oxygen-based oxidation agent used in step (iii) is selected from oxygen, air and oxygen/nitrogen mixtures different from air).

E.46. The process according to embodiment E.41, where step (iii) is carried out at a temperature of from 40 to 100° C. (especially if the oxygen-based oxidation agent used in step (iii) is hydrogen peroxide).

E.47. The process according to embodiment E.46, where step (iii) is carried out at a temperature of from 50 to 100° C. (especially if the oxygen-based oxidation agent used in step (iii) is hydrogen peroxide).

E.48. The process according to embodiment E.47, where step (iii) is carried out at a temperature of from 50 to 90° C. (especially if the oxygen-based oxidation agent used in step (iii) is hydrogen peroxide).

E.49. The process according to any of the preceding embodiments, where in step (iv) the reaction mixture obtained in step (iii) is adjusted to a pH of from 5 to 6.

E.50. The process according to any of the preceding embodiments, where in step (iv) the same basic potassium salt as in step (i) is used.

E.51. The process according to any of the preceding embodiments, for preparing potassium iron(III) ethylenediaminetetraacetic acid salt in form of granules, further comprising
(v) subjecting the reaction product of step (iv) to a granulation step.

E.52. The process according to embodiment E.51, where granulation is carried out under fluidized bed conditions.

Preferably, in step (i), the basic potassium salt is used in an amount of from 0.7 to 1 mol, more preferably from 0.7 to 0.9 mol, even more preferably from 0.75 to 0.85 mol, in particular from 0.80 to 0.82 mol, per mol of the acid form of ethylenediaminetetraacetic acid. The amount of the basic potassium salt thereby refers to the amount of potassium in said salt. Thus, if for instance a potassium salt of a twice negatively charged anion is used as a base (thus containing 2 mol of K per mol of salt), such as potassium carbonate, this salt is preferably used in an amount of from 0.35 to 0.5 mol, more preferably from 0.35 to 0.45 mol, even more preferably from 0.375 to 0.425 mol, in particular from 0.40 to 0.41 mol, per mol of the acid form of ethylenediaminetetraacetic acid.

When the above amounts of the basic potassium salt are used, the partially neutralized ethylenediaminetetraacetic acid obtained in step (i) is thus the monopotassium salt of ethylenediaminetetraacetic acid or a mixture of the monopotassium salt of ethylenediaminetetraacetic acid and the acid form of ethylenediaminetetraacetic acid.

Suitable basic potassium salts are preferably inorganic. Among these, preference is given to bases which do not introduce foreign anions into the reaction mixture. Examples of such bases are potassium hydroxide (KOH), potassium carbonate (K₂CO₃; carbonate can be converted into carbonic acid, which decomposes to water and CO₂ and the latter can be removed physically, e.g. under heating), and potassium oxide. Due to a better solubility, potassium hydroxide and potassium carbonate are preferred. Most efficient, since very well soluble in aqueous medium, very basic and not requiring any further step for its removal, and thus most preferred is however potassium hydroxide.

Step (i) is preferably carried out by preparing a suspension of the acid form of ethylenediaminetetraacetic acid in water and adding the basic potassium salt to said suspension.

The suspension of the acid form of ethylenediaminetetraacetic acid in water preferably contains the EDTA in an amount of from 0.1 to 10 mol per kg of water, more preferably from 0.5 to 5 mol per kg of water, even more preferably from 1 to 5 mol per kg of water, in particular 2 to 4 mol per kg of water. Since the potassium ethylenediaminetetraacetic acid salt formed by partial neutralization with the basic potassium salt in step (i) as well as the potassium iron ethylenediaminetetraacetic acid complex salts formed in steps (ii), (iii) and (iv) are relatively well soluble (better, for example, than the respective ammonium or sodium salts), the ethylenediaminetetraacetic acid concentration in step (i) can be relatively high, thus allowing to reduce the overall amount of waste water and to reduce energy consumption necessary for removing water to obtain the desired KFeEDTA in solid form. Thus, in a particular embodiment, the suspension of the acid form of ethylenediaminetetraacetic acid in water contains the EDTA in an amount of from 2 to 10 mol per kg of water, more preferably from 2 to 5 mol per kg of water, even more preferably from 3 to 5 mol per kg of water or from 3 to 4 mol per kg of water.

The basic potassium salt can be added in substance or as aqueous solution, but is preferably added as an aqueous solution. This eases control of exothermy and ensures proper and speedy distribution in the reaction mixture, which is of specific importance when the reaction is carried out on industrial scale. The concentration of the potassium salt in said aqueous solution is not critical, but if the concentration of ethylenediaminetetraacetic acid is to stay in more concentrated ranges, it is preferable to use relative highly concentrated solutions of the potassium salt, such as aqueous solutions containing 30-70% by weight or 40 to 60% by weight of the potassium salt, relative to the total weight of the solution.

The basic potassium salt is preferably added continually or portion-wise, the addition rate being generally thus that exothermy can be controlled.

The partial neutralization of the acid form of ethylenediaminetetraacetic acid in step (i) is generally exothermic. The reaction mixture might be cooled during the addition of the basic potassium salt, but this is not necessary if the addition form and rate of the basic potassium salt is thus that exothermy can be controlled. It might even be advantageous not to cool the reaction mixture in step (i) (as far as exothermy can be controlled, of course), but to use the reaction heat in the next step, which is preferably carried out at elevated temperature, as will be explained below.

The (partial) neutralization reaction occurs instantaneously, and thus, after all the basic potassium salt has been added, the reaction mixture can be subjected to the next step (ii), if desired after cooling (if the neutralization reaction has not been cooled), and/or if desired after isolation of the partially neutralized ethylenediaminetetraacetic acid.

The reaction mixture obtained in step (i) containing the partially neutralized ethylenediaminetetraacetic acid is reacted in step (ii) with a Fe₃O₄ source. While it is principally possible to isolate the partially neutralized ethylenediaminetetraacetic acid from the reaction mixture obtained in step (i) and then submit it to further reaction with a Fe₃O₄ source, this is neither necessary nor economical. Therefore, the reaction mixture is preferably used in step (ii) as obtained in step (i) without any intermediate purification/isolation step.

Fe₃O₄ is a mixed Fe(II) and Fe(III) oxide and can be defined more illustratively as Fe²⁺(Fe³⁺)₂O₄.

Suitable Fe₃O₄ sources are for example natural magnetite and synthetic magnetite. Synthetic magnetites are essentially pure Fe₃O₄, whereas natural magnetites might contain minor amounts of impurities (common impurities being Mg, Zn, Mn, Ni, Cr, Ti, V, Al and/or silica). Since, however, natural magnetites generally nevertheless meet the demands of the present process, and due to their lower costs as compared to synthetic magnetites, they are preferably used as Fe₃O₄ source.

The Fe₃O₄ source is preferably used in an amount of from 0.2 to 0.5 mol, more preferably from 0.2 to 0.4 mol, even more preferably from 0.30 to 0.35 mol, particularly preferably of ca. 0.33 mol, per mol of the acid form of ethylenediaminetetraacetic acid (i.e. per mol of the amount of EDTA introduced into step (i)), where the amount of the Fe₃O₄ source refers to the amount of Fe₃O₄ contained in said source. “ca.” means to include some deviation from the exact figure, due, for example to weighing errors. The deviation does generally not exceed ±10%, preferably ±5%.

Step (ii) is preferably carried out at a temperature of from 80 to 100° C., more preferably from 90 to 100° C.

Step (ii) is preferably carried out at a pH of from 3 to 5, more preferably from 3 to 4.5. Generally, this pH is obtained intrinsically by the just partial neutralization of EDTA in step (i), and thus no further measure for adjusting the pH is necessary. Should exceptionally a pH adjustment be necessary, to avoid the introduction of foreign ions, the desired pH range is preferably set by adding either additional EDTA (acid form thereof) if the pH is too basic, or the basic potassium salt used in step (i) if the pH is too acidic. The pH adjustment can take place before or during the reaction with the Fe₃O₄ source; e.g. before, during or shortly after the Fe₃O₄ source is added.

Step (ii) is preferably carried out by adding the Fe₃O₄ source to the reaction mixture obtained in step (i). Since the reaction is not exothermic, the Fe₃O₄ source can be added all at once, but also continually or portion-wise. The Fe₃O₄ source can be added in substance or as aqueous suspension, but is preferably added in substance. During or after addition of the Fe₃O₄ source, the reaction mixture is brought to the desired temperature.

If the partially neutralized ethylenediaminetetraacetic acid has been isolated before being subjected to the reaction in step (ii), this is preferably first dissolved in water before being mixed with the Fe₃O₄ source, or the partially neutralized ethylenediaminetetraacetic acid and the Fe₃O₄ source are first mixed and then dissolved/suspended in water.

The reaction in step (ii) leads to a reaction mixture containing, in addition to the desired potassium salt of Fe(III) EDTA complex, also the potassium salt of Fe(II) EDTA complex.

In step (iii), Fe(II) in said complex compounds is oxidized to Fe(III).

To this purpose, the reaction product of step (ii) is reacted with an oxygen-based oxidation agent.

While it is principally possible to isolate the potassium salt of the mixed Fe(III)/(Fe(II)-EDTA complex from the reaction mixture obtained in step (ii) and/or to remove unreacted Fe₃O₄ source, if present (removal thereof can be carried out, for example, by filtration or sedimentation), and then submit the isolate or the purified reaction mixture to oxidation step (iii), this is neither necessary nor economical. Therefore, the reaction mixture is preferably used in step (iii) as obtained in step (ii) without any intermediate purification/isolation step.

The oxygen-based oxidation agent is preferably selected from oxygen, air, oxygen/nitrogen mixtures different from air (such mixtures generally contain a higher oxygen concentration than air), and hydrogen peroxide. More preference is given to oxygen, air and hydrogen peroxide. Even more preference is given to air and oxygen.

While oxygen is a very effective oxidation agent, on industrial scale, precautionary measures might be necessary for the oxygen-rich exhaust gas, which might need to be diluted or depleted of oxygen before being released into the atmosphere. Moreover, since usual lubricant oils and greases can self-ignite in oxygen-rich atmosphere, either specific lubricants or specific non-lubricated apparatuses are needed (the same applies if oxygen-rich oxygen/nitrogen mixtures are used as oxidizing agent).

Hydrogen peroxide, generally used as aqueous solution containing e.g. ca. 10, 20, 30, 40 or 50% of H₂O₂, is also an efficient oxidation agent, but precautionary measures are necessary as well, for example to control exothermy during the oxidation process.

Surprisingly, air has proven to be effective as well. Since it has none of the problems which might arise with the use of the other oxidizing agents mentioned above, and due to its significantly lower costs, more preference is given to using air as oxidizing agent in step (iii).

If a gaseous oxidizing agent is used, such as oxygen, air or oxygen/nitrogen mixtures different from air, step (iii) can be carried out by usual means, such as sparging the reaction mixture obtained in step (b) with said gas. The gas is generally used in com-pressed form.

If hydrogen peroxide is used as oxidizing agent, step (iii) can be carried out by usual means, such as adding to the reaction mixture obtained in step (ii) an aqueous solution of hydrogen peroxide. The addition can be carried out continually or portion-wise. The addition rate is generally such that exothermy can be controlled.

Step (iii) is preferably carried out at a pressure of from atmospheric pressure to 15 bar (1.5 MPa), more preferably from atmospheric pressure to 10 bar (1 MPa). Atmospheric pressure means the local ambient pressure, and thus roughly 1013.25 hPa±200 hPa (1013.25 mbar±200 mbar).

In a more preferred embodiment, step (iii) is carried out at atmospheric pressure.

In another more preferred embodiment, step (iii) is carried out at a pressure of from 2 to 15 bar (0.2 to 1.5 MPa), more preferably from 3 to 15 bar (0.3 to 1.5 MPa), even more preferably from 3 to 10 bar (0.3 to 1 MPa), e.g. at from 4 to 10 bar (0.4 to 1 MPa) or at from 3 to 6 bar (0.3 to 0.6 MPa) or at from 3 to 5 bar (0.3 to 0.5 MPa) or at from 4 to 6 bar (0.4 to 0.6 MPa).

If hydrogen peroxide is used as oxidizing agent, the pressure is generally atmospheric pressure.

If oxygen or oxygen-rich oxygen/nitrogen mixtures, the latter containing for example 50% by weight or more of oxygen, are used as oxidizing agent, the reaction pressure is not critical and can range anywhere between atmospheric pressure and 15 bar (1.5 MPa) (and of course higher pressures, but this is neither necessary nor economic).

If air is used as oxidizing agent, the reaction pressure can also range anywhere between atmospheric pressure and 15 bar (1.5 MPa) (and of course higher pressures, but this is neither necessary nor economic), but oxidation occurs more efficiently at higher pressures, e.g. at from 2 to 15 bar (0.2 to 1.5 MPa), or at from 3 to 15 bar (0.3 to 1.5 MPa), or at from 4 to 15 bar (0.4 to 1.5 MPa), or at from 3 to 10 bar (0.3 to 1 MPa), or at from 4 to 10 bar (0.4 to 1 MPa), or at from 4 to 6 bar (0.4 to 0.6 MPa) or at from 3 to 5 bar (0.3 to 0.5 MPa). Thus, in case of using air as oxidizing agent, step (iii) is preferably carried out at a pressure above ambient pressure, preferably at from 2 to 15 bar (0.2 to 1.5 MPa), more preferably at from 3 to 15 bar (0.3 to 1.5 MPa), even more preferably at from 3 to 10 bar (0.3 to 1 MPa), in particular at from 3 to 6 bar (0.3 to 0.6 MPa), e.g. at from 4 to 6 bar (0.4 to 0.6 MPa) or at from 3 to 5 bar (0.3 to 0.5 MPa).

While the reaction temperature in step (iii) can principally be as high as the temperature of step (ii), the suitable reaction temperature ranging e.g. from 10 to 100° C., it has been found that oxidation rates are higher at lower reaction temperatures if a gaseous oxidizing agent, such as oxygen, air or oxygen/nitrogen mixtures different from air are used, and decomposition of the resulting products is also reduced. Thus step (iii) is more preferably carried out at from 10 to 40° C., even more preferably from 15 to 30° C., in particular from 20 to 30° C. or from 20 to 25° C., especially if gaseous oxidizing agents are used. It is however also possible to carry out step (iii) at higher temperatures, such as 40 to 100° C. or 50 to 100° C. or 50 to 90° C., e.g. in order to shorten the process time.

In case of hydrogen peroxide, the reaction temperature is preferably in the range of from 10 to 100° C., more preferably from 50 to 100° C. and even more preferably from 50 to 90° C., e.g. from 70 to 90° C.

If during step (iii) the amount of water is depleted, which can occur, for example, if a gaseous oxidizing agent, such as air or oxygen, is sparged through the reaction mixture and entrains water, it is advisable to replace the depleted water to avoid the formation of precipitates due to an up-concentration of reactants and products. The replacement of water can be carried out during step (iii) or, more conveniently, after completion of step (iii), e.g. before, during or after adjustment of the pH in step (iv).

The pH of the reaction mixture obtained in step (iii) is generally slightly acidic, and thus, in step (iv), the reaction mixture obtained in step (iii) is generally adjusted to a pH of from 5 to 7, preferably to a pH of 5 to 6, by mixing the reaction mixture obtained in step (iii) with a base, preferably by adding a base to the reaction mixture obtained in step (iii). Suitably, to avoid the introduction of foreign cations or other foreign materials, the base is a basic potassium salt, preferably the same basic potassium salt as used in step (i). Here, too, the basic potassium salt is preferably added as an aqueous solution.

The basic potassium salt base is preferably added in amount to obtain a fully neutralized KFe(III)EDTA, i.e. in such an amount that the molar ratio of the total amount of the basic potassium salt used in steps (i) and (iv) and ethylenediaminetetraacetic acid used in step (i) is ca. 1:1. If in step (i) the basic potassium salt has been added in an amount of 1 mol (or more) per mol of ethylenediaminetetraacetic acid, the pH might already be in the desired range, and pH adjustment is not necessary. In this case, step (iv) might be skipped. “ca.” in this case, too, means to allow a deviation of ±10%, preferably ±5% from the exact ratio.

Suitable reaction vessels for carrying out steps (i) to (iv) are known to the skilled person. If no intermediate isolation or purification step is carried out between the different reaction steps (i) to (iv), the reaction in steps (i) to (iv) can be carried out in the same reaction vessel; where in case of using oxygen or oxygen-rich oxygen-nitrogen mixtures as oxidizing agents the above-mentioned conditions (specific lubricant-free apparatus or specialty lubricant) have to be applied. Suitable reactors contain at least a means for introducing liquid and solid reactants, and, if applicable, a means for introducing gaseous oxidizing agents as well as a gas outlet (in case of oxygen or oxygen-rich oxygen-nitrogen mixtures as oxidizing agents the gas outlet has to be connected to suitable equipment for depleting the exhaust gas in oxygen and/or returning it to the reaction). Moreover, the reactor expediently contains a stirrer and means for cooling and/or heating; expedient is also the presence of a sensor for pH control.

Steps (i) to (iv) can alternatively be carried out in different reaction vessels, each adapted to the specific needs of the respective step.

If desired, the product obtained in step (iv) can be subjected to further purification, such as filtration, if necessary after dilution, to remove solid residues stemming, for example, from the Fe₃O₄ source, especially if this is a natural magnetite, or undesired by-products.

To obtain a solid KFe(III)EDTA product, standard procedures can be applied, such as crystallization, if necessary after concentrating the aqueous product, e.g. by partial evaporation of water, or total removal of water, e.g. by total evaporation, if desired under reduced pressure, or by spray drying.

Preferably, however, the KFe(III)EDTA is converted into granules.

Thus, in a preferred embodiment, the process of the invention is one for preparing potassium iron(III) ethylenediaminetetraacetic acid salt in form of granules, further comprising
(v) subjecting the reaction product of step (iv) to a granulation step.

Granulation can be carried out by known methods, such as granulation in a high-shear granulator, in a twin screw granulator or in a fluidized bed granulator. Preferably, granulation is carried out under fluidized bed conditions.

The process of the invention leads to the desired KFe(III)EDTA in good yields, with high total iron content, low Fe(II) content, does not require any catalyst and avoids the presence of undesired foreign anions such as sulfate, chloride or nitrate. Moreover, the process can be carried out with low-priced materials, such as natural magnetite as iron source and air as oxidizing agent, and is thus economic and efficient.

The following examples serve as further illustration of the invention.

### EXAMPLES

### Analytics

To monitor the progress of the reaction and conversion of EDTA into the potassium salt of the iron complex, the quantity of free EDTA (i.e. acid form) was determined via titration with a 0.05 mol Fe₂(SO₄)₃ solution.

The amount of Fe²⁺ and Fe³⁺ in the KFe(III)EDTA product were also determined titrimetrically:

Fe²⁺ determination: Into a heated titration vessel at 80° C. were added 60 ml of 1 N HCl solution. A weighted sample of the reaction solution was added and titrated with a 0.1 mol K₂Cr₂O₇ solution.

Fe³⁺ determination: Into a heated titration vessel at 80° C. were added 60 ml of 1 N HCl solution. A weighted sample of the obtained reaction solution was added and titrated with a 0.1 mol TiCl₃ solution.

### Example 1: Synthesis of KFe(III)EDTA—Oxidation with Air at Atmospheric Pressure

a) 1369.7 g (76.032 mol) of water were placed in a 4 l three-necked flask equipped with stirrer and cooler. 1 kg (3.422 mol) of EDTA (acid form; Trilon® BS, from BASF SE) was added via a powder funnel. A suspension was formed without any exotherm. Under stirring, 311.0 g (2.772 mol, 0.8 mol eq.) of an aqueous 50% KOH solution was added dropwise within 30 minutes. Since the reaction of KOH with EDTA is strongly exothermic, the reaction suspension was cooled with ice during the addition. However, analogous experiments without cooling showed that this is not mandatory and the quality of the EDTA potassium partial salt formed is not affected by higher temperatures.

b) Under stirring, 261.5 g (1.129 mol) of a natural magnetite (Magnachem® WT-1 from LKAB, Sweden) were added via a powder funnel. The stirred reaction mixture was heated to 95° C. and kept at this temperature for 5 hours. During the heating, a dark red-brownish solution with a pH of around 3-4 was formed with small amounts of solid parti-des of unreacted magnetite. The product suspension was allowed to cool to room temperature.

c) A strong air stream was now passed via a frit to the solution for 4 hours.

d) During the oxidation step, some water was stripped out by the airflow and this loss was replaced. The suspension was then adjusted to a pH of 5.5 by the addition of 51 ml of 50% (0.45 mol, 0.2 mol eq.) KOH.

Finally, the reaction solution was filtrated via a suction filter to give clear solution of KFe(III)EDTA.

Yield: 3335 g solution containing 1297.8 g of the desired KFe(III)EDTA

(C₁₀H₁₂FeN₂O₈K; M=383.15 g/mol), 99% yield relative to the EDTA starting material.

Total Fe content in KFeEDTA: 14% with Fe²⁺~0.2-<1.0%.

The solution was analyzed by electrospray mass spectroscopy (ESI).

MS: Mr 344 [M]⁻ (C₁₀H₁₂O₈N₂Fe)

The obtained KFe(III)EDTA solution could be directly used for granulation.

### Example 2: Synthesis of KFe(III)EDTA—Oxidation with Oxygen at Atmospheric Pressure

a) 1759 g (97.6 mol) of water were placed in a 4 l three-necked flask equipped with stirrer and cooler. 1900 g (6.5 mol) of EDTA (acid form; Trilon® BS, from BASF SE) were added via a powder funnel. A suspension was formed without any exotherm. Under stirring and cooling, 590.9 g (5.26 mol, 0.8 mol eq.) of an aqueous 50% KOH solution was added dropwise within 30 minutes, keeping the temperature below.

b) Under stirring, 602 g (2.6 mol) of a natural magnetite (Magnachem® WT-1 from LKAB) was added via a powder funnel. The stirred reaction mixture was heated to 95° C. and kept at this temperature for 5 hours. The product suspension was allowed to cool to 20° C.

c) An aliquot of 1 l of this reaction mixture was placed into a 3-neck round-bottomed flask equipped with a reflux condenser, thermometer, and an oxygen inlet (dipped glass tube). The equipment must be free of oil and lubricating grease. Then oxygen was passed through the solution for three hours at a flow rate of 30 L/h and an internal temperature of 20-25° C.

d) The suspension was then adjusted to a pH of 5.5 by the addition of KOH.

Filtration of this reaction suspension through a suction filter gave a clear red-brown solution containing KFe(III)EDTA.

Yield: 99% relative to the EDTA starting material.

Total Fe content in KFeEDTA: 14.5% with Fe²⁺ <0.2%.

This obtained KFe(III)EDTA solution could be directly used for granulation.

### Example 3: Synthesis Procedure for KFe(III)EDTA—Oxidation with Pressurized Air

200 ml of a reaction mixture obtained in analogy to example 2b) was placed given in an autoclave. Pressurized air at p=4 bar was passed through the autoclave for 4 hours at 20° C. The composition of air after passing the reaction was monitored. When no more uptake of oxygen could be measured, the oxidation was stopped. The oxidation with pressurized air was also performed at 50° C. and 80° C., the reaction rates were however prolonged as compared to the reaction at 20° C.

The suspension was then adjusted to a pH of 5.5 by the addition of KOH. Filtration of this reaction suspension through a suction filter gave a clear red-brown solution which was directly used for granulation.

Yield: 99% relative to the EDTA starting material.

Total Fe content of KFe(III)EDTA (100% active): 14% with Fe^{2+~0.2}-0.3%.

This obtained KFe(III)EDTA solution could be directly used for granulation.

### Example 4: Synthesis Procedure for KFe(III) EDTA—Oxidation with Hydrogen Peroxide

1.0 l of a reaction mixture obtained in analogy to example 2b) was placed into a 3-neck round-bottomed flask equipped with a reflux condenser, thermometer, and dropping funnel and heated to 80° C. under stirring. 33.6 mL of a 30 wt.-% aqueous solution of hydrogen peroxide (0.55 mol per mol of Fe^{II}) was added within 1 hour. Then the reaction mixture was allowed to cool to room temperature and the suspension was then adjusted to a pH of 5.5 by the addition of KOH. Filtration of this reaction suspension through a suction filter gave a clear red-brown solution.

This obtained KFe(III)EDTA solution could be directly used for granulation.

### Example 5: Synthesis of KFe(III)EDTA—Reaction with a Synthetic Magnetite and Oxidation with Air at Atmospheric Pressure

a) 1369.7 g (76.032 mol) of water were placed in a 4 l three-necked flask equipped with stirrer and cooler. 1 kg (3.422 mol) of EDTA (acid form; Trilon® BS, from BASF SE) was added via a powder funnel. A suspension was formed without any exotherm. Under stirring, 311.0 g (2.772 mol, 0.8 mol eq.) of an aqueous 50% KOH solution was added dropwise within 30 minutes. Since the reaction of KOH with EDTA is strongly exothermic, the reaction suspension was cooled to <20° C. with ice during the addition.

b) Under stirring, 261.5 g (1.129 mol) of a synthetic magnetite (Bayferrox® 318 M from Lanxess) were added via a powder funnel. The stirred reaction mixture was heated to 95° C. and kept at this temperature for 3 hours. The product suspension was allowed to cool to room temperature.

c) A strong air stream was now passed via a frit to the solution for 8 hours.

d) During the oxidation step, some water was stripped out by the airflow and this loss was replaced. Finally, the reaction solution was filtrated via a suction filter and the suspension was then adjusted to a pH of 5 by the addition of 50% KOH. to give clear solution of KFe(III)EDTA.

Yield: 83.5% relative to the EDTA starting material.

Total Fe content in KFeEDTA: 13% with Fe² 0.1%.

### Example 6: Granulation of KFe(III)EDTA

The granulation was performed on a DMR granulation equipment. As template, KFe(III)EDTA, which had previously been completely dried in a drying oven for 24 h at 95° C. and then ground in a laboratory mill (Polymix PX-MFC 90 D), was used. For granulation, 150 mL of a saturated KFe(III)EDTA solution was placed in an open stirred vessel and continuously agitated with an anchor stirrer. With a pump rate of 17-19 g/min, the solution was conveyed to the nozzle (diameter of 1 mm) in the granulation apparatus. The nozzle pressure during the entire granulation was p=1.5 bar (atomization pressure), the pressure of the purge gas p=2.0 bar. The differential pressure was detected as p=27 mbar, the differential pressure in the filter was p=10 mbar, but it in-creased to 16 mbar during the course of the experiment. The product temperature was set to T=100° C. with a supply air temperature of T=170° C. The gas pressure during granulation was p=2.1 bar and the gas flow rate was 25 m³/h. Nitrogen was used as the gas atmosphere. The material was subjected to sieving after granulation was complete. 26 g of stable granules were obtained.

1. A process for preparing potassium iron(III) ethylenediaminetetraacetic acid complex salt KFe(III)EDTA, comprising
(i) reacting in an aqueous medium the acid form of ethylenediaminetetraacetic acid with a basic potassium salt to a partially neutralized ethylenediaminetetraacetic acid;
(ii) reacting the reaction mixture obtained in step (i) with a Fe₃O₄ source to a potassium iron ethylenediaminetetraacetic acid complex salt containing both iron (II) and (III);
(iii) oxidizing the potassium iron ethylenediaminetetraacetic acid complex salt containing both iron (II) and (III) to potassium iron(III) ethylenediaminetetraacetic acid complex salt with an oxygen-based oxidation agent; and
(iv) adjusting the reaction mixture obtained step (iii) to a pH of from 5 to 7 with a basic potassium salt.

2. The process according to claim 1, wherein in step (i) the basic potassium salt is used in an amount of from 0.7 to 1 mol per mol of the acid form of ethylenediaminetetraacetic acid; wherein the amount of the basic potassium salt refers to the amount of potassium in said salt; and wherein the partially neutralized ethylenediaminetetraacetic acid obtained in step (i) is the monopotassium salt of ethylenediaminetetraacetic acid or a mixture of the monopotassium salt of ethylenediaminetetraacetic acid and the acid form of ethylenediaminetetraacetic acid.

3. The process according to claim 1, wherein the basic potassium salt used in steps (i) and (iv) is KOH.

4. The process according to claim 1, wherein the Fe₃O₄ source is natural or synthetic magnetite.

5. The process according to claim 4, wherein the Fe₃O₄ source is natural magnetite.

6. The process according to claim 1, wherein the Fe₃O₄ source is used in an amount of from 0.2 to 0.5 mol per mol of the acid form of ethylenediaminetetraacetic acid, wherein the amount of the Fe₃O₄ source refers to an amount of Fe₃O₄ contained in said source.

7. The process according to claim 1, wherein step (ii) is carried out at a temperature of from 80 to 100° C.

8. The process according to claim 1, wherein step (ii) is carried out at a pH of from 3 to 5.

9. The process according to claim 1, wherein step (iii) is carried out at a pressure of from atmospheric pressure to 15 bar.

10. The process according to claim 9, wherein step (iii) is carried out at atmospheric pressure; or is carried out at a pressure of from 3 to 10 bar.

11. The process according to claim 1, wherein the oxygen-based oxidation agent used in step (iii) is selected from the group consisting of oxygen, air, oxygen/nitrogen mixtures different from air, and hydrogen peroxide.

12. The process according to claim 11, wherein the oxygen-based oxidation agent used in step (iii) is selected from the group consisting of oxygen, air and hydrogen peroxide.

13. The process according to claim 12, wherein the oxygen-based oxidation agent in step (iii) is air.

14. The process according to claim 1, wherein step (iii) is carried out at a temperature of from 10 to 100° C.

15. The process according to claim 1, wherein
the oxygen-based oxidation agent used in step (iii) is selected from the group consisting of oxygen, air and oxygen/nitrogen mixtures different from air, and step (iii) is carried out at a pressure of from atmospheric pressure to 15 bar; or
the oxygen-based oxidation agent in step (iii) is hydrogen peroxide, and step (iii) is carried out at atmospheric pressure and a temperature of from 40 to 100° C.

16. The process according to claim 1, wherein in step (iv) the reaction mixture obtained in step (iii) is adjusted to a pH of from 5 to 6.

17. The process according to claim 1, for preparing potassium iron(III) ethylenediaminetetraacetic acid complex salt in form of granules, further comprising
(v) subjecting the reaction product of step (iv) to a granulation step.

18. The process according to claim 17, wherein granulation is carried out under fluidized bed conditions.

19. The process according to claim 2, wherein in step (i) the basic potassium salt is used in an amount of from 0.7 to 0.9 mol per mol of the acid form of ethylenediaminetetraacetic acid.

20. The process according to claim 3, wherein the KOH is added as an aqueous solution.
